(19) United States
(12) Patent Application Publication
(10) Pub. No.: US 2008/0266117 A1
(43) Pub. Date: Oct. 30, 2008

(54) SENSORS AND DISPOSABLE ARTICLES THAT CONTAIN THE SENSORS

(76) Inventors: Xuedong Song, Roswell, GA (US); James Matthew Takeuchi, Roswell, GA (US); Kaiyuan Yang, Cumming, GA (US); Shawn Jeffery Sullivan, Neenah, WI (US); Sridhar Ranganathan, Suwanee, GA (US)

Correspondence Address: Christopher M. Goff (27839);ARMSTRONG TEASDALE LLP, ONE METROPOLITAN SQUARE, SUITE 2600, ST. LOUIS, MO, 63102, US

(21) Appl. No.: 11/742,448

(22) Filed: Apr. 30, 2007

(51) Int. Cl. G08B 23/00; A61F 13/15

(52) U.S. Cl. 340/573.5; 604/361

(57) ABSTRACT

Embodiments of the invention provide a sensor for detecting the presence of fluid in an absorbent article. The sensor may include a fluid activated battery. Fluid received in the absorbent article may connect electrodes of the fluid activated battery and cause a voltage to be generated between battery electrodes. The voltage generated between the electrodes may provide power to the sensor circuit. In one embodiment, the fluid activated battery may be configured to detect the presence of fluid in the absorbent article and the presence and/or amount of particular substances in the received fluid.

### BACKGROUND OF THE INVENTION

### Description of the Related Art

Fluid excretion from the human body may provide several indications regarding the health of a person. For example, the amount and rate of excretion of fluid, for example, urine, may determine whether a person is well hydrated. The urine excreted by a person may also provide indication of disease conditions, glucose concentration in a person, and the like.

Data derived from the analysis of fluid excretion may be critical to making health decisions regarding a person. For example, determining the hydration of a person may be critical to providing proper care to persons who may not be able to care for themselves. Providing proper care may involve providing such persons with sufficient nutrition. Some of the most important nutrients are fluids, for example, water. Water plays a vital role in regulating body temperature, transporting other nutrients and oxygen to cells, removing waste, cushioning joints, protecting organs and tissues, and many other significant biological functions. Therefore, keeping a person well hydrated is vital to maintaining the health of the person.

Determining hydration may be especially critical while caring for newborns that are unable to communicate with a caregiver. For example, it is crucial for a newborn to get sufficient nutrition in the first few weeks to ensure proper development. In the case of breast-feeding babies, mothers have great difficulty in judging whether their babies are receiving sufficient milk. Typically, pediatricians advise parents to monitor the number of diapers that are wetted by the child per day to determine whether the child is sufficiently hydrated. In other words, pediatricians rely on the excretion of bodily fluids to determine the hydration of children.

Similarly, fluid excretions such as urine may be critical to determining whether there are any disease conditions inflicting a person. For example, the glucose concentration in urine may be related to the status of glucose in blood. Therefore, by studying fluid excretions from the body to determine a person's disease, appropriate decisions regarding, for example, the nature of treatment, diet, and the like may be facilitated.

Fluid excretion such as urine may be collected in an absorbent product such as a diaper for some persons, such as, for example, newborns and the elderly. Modern diapers typically include an electronic sensor to detect wetness. When a wetting event occurs, the fluid received in the diaper may be detected by an electronic sensor and the sensor may generate a signal indicating the presence of fluid in the diaper. By facilitating detection of wetting events, the electronic sensor may aid caretakers in potty training, monitoring hydration status of a child, etc.

Prior art sensors typically require a constant source of power to operate the sensor circuit, even when no fluid is present in the diaper. Therefore, batteries are typically included in the diapers to provide power. However, including batteries in disposable products may not be cost effective. Furthermore, even if no fluid is present in the diaper, a battery may self discharge, thereby making the battery unreliable and incapable of providing sufficient power to the sensor. Furthermore, current absorbent articles provide no capability of detecting the characteristics of the bodily fluid received in the absorbent article that may be used to analyze specific health conditions.

Therefore, what is needed are improved methods, systems, and articles of manufacture to power a sensor circuit in an absorbent product and determine characteristics of the fluid received therein.

### SUMMARY OF THE INVENTION

The present invention generally relates to absorbent articles, and more specifically to sensors configured to detect fluids and one or more characteristics of a fluid in absorbent articles.

One embodiment of the invention provides an apparatus, generally comprising an absorbent article and a sensor circuit disposed on the absorbent article and configured for detecting fluid in the absorbent article. The sensor circuit generally comprises a pair of sensor electrodes, wherein each electrode is coupled with an absorbent material, the absorbent material being configured to receive fluid to electrically couple the sensor electrodes and a processing circuit coupled with the sensor electrodes and configured to detect the presence of fluid that electrically couples the sensor electrodes. The sensor circuit further comprises a battery for providing power to the processing circuit, wherein the battery is configured to be activated by the fluid received in the absorbent article.

Another embodiment of the invention provides an apparatus, generally comprising an absorbent article and a sensor circuit disposed on the absorbent article. The sensor circuit generally comprises a battery comprising at least two electrodes coated with a material for detecting the presence of analytes in fluid received in the absorbent article, wherein the battery is activated by fluid received in the absorbent article, and a processing circuit, wherein the processing circuit is configured to receive electric power from the battery when the battery is activated, and generate a signal indicating presence of one or more predetermined analytes in the fluid received in the absorbent article.

Yet another embodiment of the invention provides a method for detecting the presence of fluid in an absorbent article. The method generally comprises disposing a pair of battery electrodes in an absorbent area of the absorbent article, wherein the battery electrodes are configured to generate electrical power in response to receiving fluid in the absorbent article, and disposing a processing circuit configured to receive the electrical power from the battery electrodes and generate a signal indicating presence of one or more analytes in the fluid received in the absorbent article.

A further embodiment of the invention provides an apparatus, generally comprising an absorbent article and a sensor circuit disposed on the absorbent article. The sensor circuit generally comprises a battery comprising at least two electrodes separated by an absorbent material comprising a material for detecting the presence of analytes in fluid received in the absorbent article, wherein the battery is activated by fluid received in the absorbent article, and a processing circuit, wherein the processing circuit is configured to receive electric power from the battery when the battery is activated and generate a signal indicating presence of one or more predetermined analytes in the fluid received in the absorbent article.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments thereof which are illustrated in the appended drawings.

It is to be noted, however, that the appended drawings illustrate only typical embodiments of this invention and are therefore not to be considered limiting of its scope, for the invention may admit to other equally effective embodiments.

FIG. 1A illustrates an exemplary absorbent article according to an embodiment of the invention.

FIG. 1B illustrates an exemplary processing circuit according to an embodiment of the invention.

FIG. 2 illustrates an exemplary sensor circuit according to an embodiment of the invention.

FIG. 3 illustrates an exemplary fluid activated battery according to an embodiment of the invention.

FIG. 4 illustrates another exemplary sensor circuit according to an embodiment of the invention.

### DETAILED DESCRIPTION OF THE PREFERRED EMBODIMENTS

Embodiments of the invention provide a sensor circuit for detecting the presence of fluid and characteristics of the fluid in an absorbent article. The sensor circuit may include a fluid activated battery. Fluid received in the absorbent article may activate the fluid activated battery and cause a voltage to be generated. The activated battery may provide power to one or more circuits in the absorbent article, for example, a wetness sensor circuit. In one embodiment, the fluid activated battery may also be configured to detect the presence and/or concentration of particular substances in the received fluid.

In the following, reference is made to embodiments of the invention. However, it should be understood that the invention is not limited to specific described embodiments. Instead, any combination of the following features and elements, whether related to different embodiments or not, is contemplated to implement and practice the invention. Furthermore, in various embodiments the invention provides numerous advantages over the prior art. However, although embodiments of the invention may achieve advantages over other possible solutions and/or over the prior art, whether or not a particular advantage is achieved by a given embodiment is not limiting of the invention. Thus, the following aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

### EXEMPLARY SYSTEM

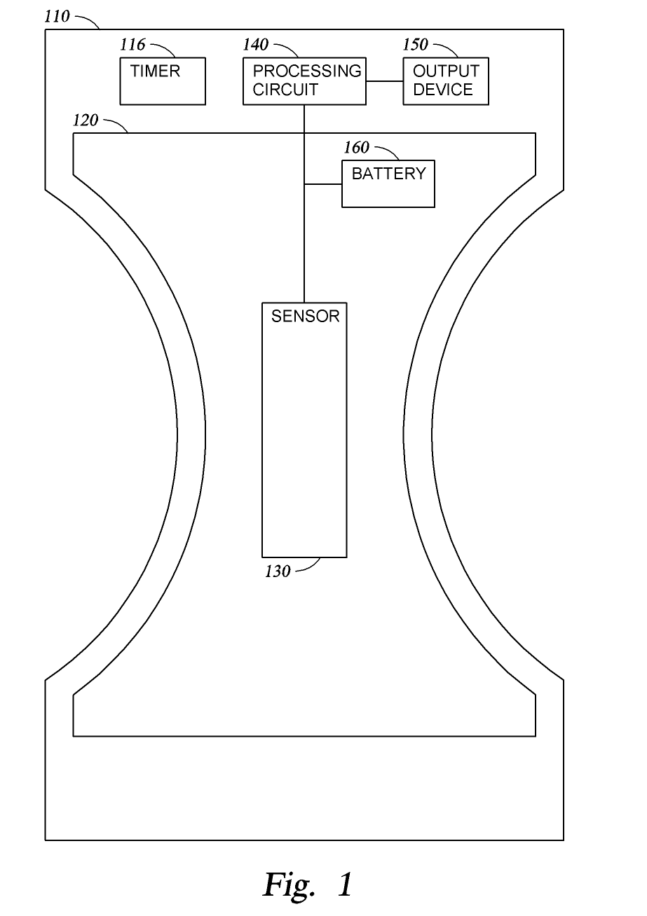

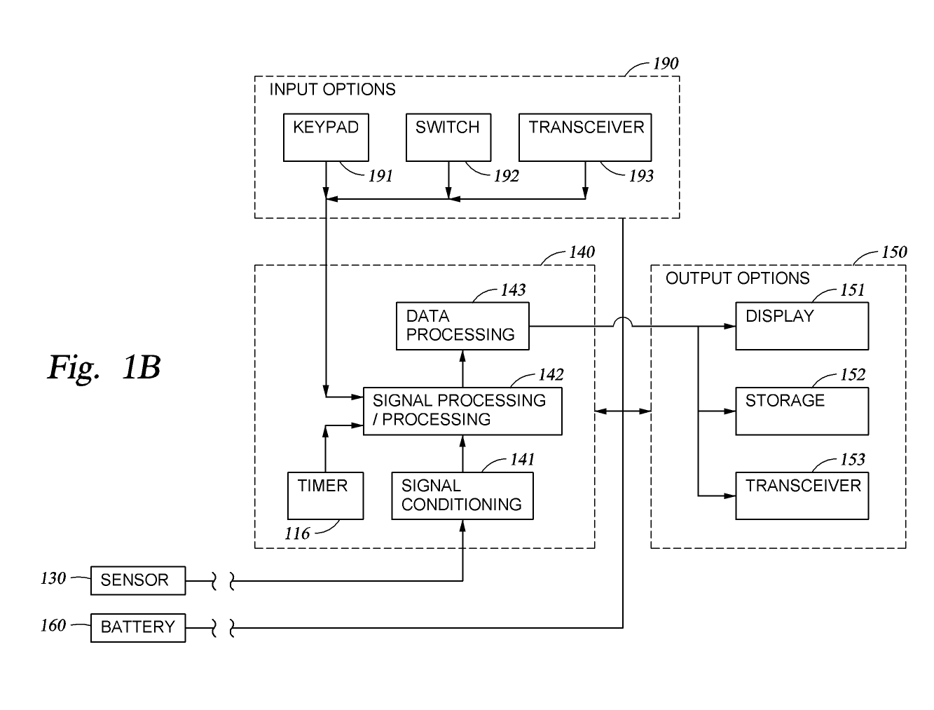

FIG. 1A illustrates an exemplary absorbent article 110 in which embodiments of the invention may be implemented. Absorbent article 110 may or may not be disposable. For purposes of illustration, absorbent article 110 is shown as a diaper. However, one skilled in the art will recognize that absorbent article 110 may include any article intended for personal wear, including, but not limited to, athletic gear, training pants, feminine hygiene products, incontinence products, medical garments, surgical pads, bandages, personal care or health care garments, and the like. More generally, absorbent article 110 may be any article configured to receive and retain fluid.

Absorbent article 110 may include an absorbent area 120, wetness sensor 130, processing circuit 140, output device 150, and battery 160. Absorbent area 120 may be made from any appropriate material configured to absorb and retain fluid. For example, absorbent area 120 may be made from cotton, synthetic polymers such as hydrogels, superabsorbents, hydrocolloids, absorbent web materials, and the like.

Sensor 130 may be disposed in the absorbent area 120. Fluid received in absorbent area 120 may alter an electrical property of the sensor. The altering of the electrical property of sensor 130 may indicate the presence of the fluid received in the absorbent area. For example, fluid received in absorbent area 120 may alter the resistance, conductance, impedance, capacitance, inductance, or the like of sensor 130. In one embodiment, the amount of a substance in the fluid in absorbent area 120 may be determined based on an amount of change in the electrical property of the sensor.

In one embodiment, sensor 130 may be configured to determine the presence of particular analytes in the fluid received in the absorbent area 120. For example, sensor 130 may be configured to determine the presence and/or concentration of particular enzymes and/or ions in the fluid. Detecting the particular enzymes and ions may facilitate diagnosis of the health status of a person wearing the absorbent article 110.

Processing circuit 140 may be coupled with sensor 130 as illustrated in FIG. 1A. Processing circuit 140 may be configured to measure an electrical or physical property of sensor 130. For example, in one embodiment, processing circuit 140 may be configured to measure an electrical resistance associated with the sensor 130. In a particular embodiment, sensor 130 may include two electrodes. Fluid received in the absorbent area 120 may connect the two electrodes of sensor 130, thereby reducing the resistance measured by the processing circuit 140, and indicating the presence of fluid in the absorbent area 120.

In one embodiment, particular analytes present in the absorbent area may affect an electrical property of the sensor 130. For example, particular analytes may enhance a current that flows between the sensor electrodes 130. Processing circuit 140 may detect the enhanced current and determine the presence and/or amount of the analyte present in the fluid. Analyte detection is described in greater detail in the following sections.

In one embodiment, an output device 150 may be included in the absorbent article 110, as illustrated in FIG. 1A. Processing circuit 140 may be configured to provide output device 150 with data associated with the presence of the fluid in the absorbent article 110. The output device 150 may be coupled permanently or detachably with absorbent article 110.

In one embodiment of the invention, output device 150 may include a display means, for example, a Liquid Crystal Display (LCD), coupled with absorbent article 110. Processing circuit 140 may be configured to display a value indicating the health status of a person wearing the absorbent article. For example, processing circuit 140 may compute and display any combination of the measured electrical or physical property of sensor 130, the amount of fluid in absorbent area 120, a rate of fluid output from a person wearing absorbent article 110, a health status of the person wearing the diaper, specific analytes detected in the fluid, and the like.

In one embodiment of the invention, output device 150 may include an array of Light Emitting Diodes (LEDs) for indicating the presence of and/or amount of fluid in the absorbent article. For example, as the absorbent article is wetted by the fluid, one or more LEDs may be lit to indicate the amount of fluid in the diaper. In one embodiment, a plurality of arrays may be provided, each array depicting a value associated with the fluid of the absorbent article or a person wearing the absorbent article. For example, a first array may indicate the fluid loading of the absorbent article, a second array may depict the number of insults, a third array may indicate a hydration status of the person wearing the diaper, and so on. An insult may be a distinct wetting event in the absorbent article. Each array may be differentiated for example, by the color of light emitted by the array. For example, red LEDs may depict loading, green LEDs may depict number of insults, and so on.

One or more predetermined LEDs may also indicate the presence and/or amount of specific analytes in the absorbent article. For example, processing circuit 140 may be configured to detect specific analytes in the fluid received in the absorbent article. If a particular analyte is detected, an LED associated with the particular analyte may be illuminated. For example, if nitrites are detected in urine received in a diaper, an LED may be illuminated to indicate the possibility of urinary tract infection.

In one embodiment, the display means may output a qualitative description of the fluid in the absorbent article or a person wearing the absorbent article. For example, the display means may output a plurality of levels of hydration ranging from, for example, “well hydrated” to “severely dehydrated,” or some equivalent scale thereof for determining hydration, thereby allowing a caregiver to take appropriate action. In one embodiment, the display means may be configured to display a suggested course of action for a caregiver based on the nature of the fluid in the absorbent article or the person. For example, the suggested course of action may include “change diaper,” “feed milk/water,” “call doctor,” and the like.

Processing circuit 140 may include logic for determining a quantitative output based on one or more parameters measured by the processing circuit. For example, in one embodiment, processing circuit 140 may determine the quantitative output based on measurements of fluid in the absorbent article and/or the detection of specific analytes in the fluid. In one embodiment, processing circuit 140 may be coupled with memory comprising data for determining the quantitative output. For example, the data may include ranges of fluid output rates, wherein each range is associated with a particular hydration recommendation for the caregiver. Processing circuit 140 may measure the fluid output rate in the absorbent article, compare the fluid output rate to the range data, and provide a quantitative output using the display means to the caregiver.

In one embodiment, processing circuit 140 may be configured to receive one or more inputs from a caregiver. A caregiver may provide, for example, age and weight profile of the wearer of the absorbent article. Processing circuit 118 may use the data inputs for calculating one or more values, for example, the fluid output rate, a recommendation for a course of action, and the like. Accordingly, one or more input devices, for example, buttons, dip switches, and the like, may be coupled with the absorbent article for facilitating data input. In one embodiment, the input device may be integrated with output device 150, for example, a touchscreen.

In one embodiment of the invention, output device 150 may include a wireless transmitter to transmit data from the absorbent article to another device, for example, a computer, cell phone, personal digital assistant, and the like. The transmitted data may include, for example, the measured electrical or physical property of sensor 130, the amount of fluid in absorbent area 120, a rate of fluid output from a person wearing absorbent article 110, a quantitative description of the health status of a person wearing the absorbent article, a suggested course of action, and the like. The wireless transmission of data from a wearable product such as an absorbent article to an external device is discussed in greater detail in the applications of Wolkowicz, et al., application Ser. No. 10/720,776; Lye, et al., application Ser. No. 10/305,263; and Drinan, et al. application Ser. No. 10/032,765. The aforementioned applications are incorporated herein by reference in their entirety.

In one embodiment, output device 150 may connect with a network, for example, a Local Area Network (LAN), Metropolitan Area Network (MAN), or a Wide Area Network (WAN) for uploading data related to the fluid received in an absorbent article. In a particular embodiment, the output device may connect with the internet for uploading data to a website configured to accumulate and analyze the data. In another particular embodiment, output device 150 may connect with a hospital LAN to upload the data. The analysis of data may include, for example, comparing received data with historic data accumulated for a user of the absorbent article. The analysis may be sent to a care giver, for example, via email, text message, and the like.

In one embodiment of the invention, output device 120 may also include a receiver for receiving wireless signals from a device, for example, a computer. In one embodiment, output device 150 may transmit data to one or more peripheral processing devices, for example, a computer, website, cell phone, and the like. The peripheral device may analyze the data and transmit one or more signals to the absorbent article. The transmitted signals may include data for display on the output device, for example, a recommended course of action for a caregiver. Accordingly, processing circuit 140 may be configured to display data received via the receiver on the display means.

Absorbent article 110 may also include a timer 116. Timer 116 may be configured to determine a time period over which fluid is received in absorbent article 110. For example, in one embodiment, timer 116 may be coupled with a diaper and may be configured to start at the time when fluid is first received in the diaper. The timer may be stopped at the time of removal of the diaper, thereby providing a time period over which fluid is received in the diaper. In one embodiment, timer 116 may be detachably coupled with absorbent article 110. Therefore, the same timer may be used to determine a time period for receiving fluid in multiple absorbent articles

Absorbent article 110 may also include a battery 160. Battery 160 may be coupled with the processing circuit 140, output device 150, or any other circuit requiring electric power. In one embodiment of the invention, the battery 160 may be a fluid activated battery. Accordingly, battery 160 may be disposed in the absorbent area 120 at a location where fluid is likely to be received, as illustrated in FIG. 1A. The fluid activated battery is described in greater detail in the following section.

FIG. 1B illustrates a more detailed view of the processing circuit 140, output device 150, and an input device 190, according to an embodiment of the invention. Processing circuit 140 may include a signal conditioning circuit 141, signal processing circuit 142 and a data processing circuit 143. In one embodiment, processing circuit 140 may include the timer 116.

Signal conditioning circuit 141 may be configured to receive one or more electrical signals from the sensor 130 and prepare the electrical signals for processing. In one embodiment of the invention, signal conditioning circuit 141 may receive an electrical signal directly from a battery 160. In one embodiment, the electrical signals received by the signal conditioning circuit 141 may indicate the presence of fluid in the absorbent area 140 and one or more characteristics of the fluid. Preparing the signals for processing may involve signal amplification, filtering, conversion, and the like. Accordingly, signal conditioning circuit may include signal filters, instrument amplifiers, sample-and-hold amplifiers, isolation amplifiers, signal isolators, multiplexers, bridge conditioners, analog-to-digital converters, digital-to-analog converters, frequency converters or translators, voltage converters or inverters, frequency-to-voltage converters, voltage-to-frequency converters, current-to-voltage converters, current loop converters, charge converters, and the like.

Signal processing circuit 142 may be configured to capture one or more data points from the electrical signals received from either one of the sensor 130 and the battery 160. For example, in one embodiment, signal processing circuit 142 may capture a rate of current flow between sensor or battery electrodes. In one embodiment of the invention, signal processing circuit may be configured to capture data from an input device 190, as illustrated in FIG. 1B. Data captured from the input device 190 may include data manually provided to the absorbent article and data wirelessly received from an external device.

Data processing circuit 143 may process the data captured by the signal processing circuit 142. For example, data processing circuit may determine whether a particular analyte is presence in fluid received in the absorbent article 110, the amount of analyte that is present, and the like, based on the data captured by the signal processing circuit 142. In one embodiment, data processing circuit 143 may be configured to display processed data using the output device 150.

FIG. 1B illustrates an output device 150 according to an embodiment of the invention. Output device 150 may include a display device 151, storage device 152, and a transceiver 153. Display device may be any one of the devices discussed above, for example, LEDs, a display screen, and the like. Storage device 152 may include a random access memory for example, a dynamic random access memory (DRAM), static random access memory (SRAM), and the like. In one embodiment, storage device 152 may exist in multiple levels, including one or more levels of cache. Storage device 152 may be configured to store data collected from the absorbent article 110, for example, data regarding one or more characteristics of fluid in the absorbent article. In one embodiment, storage device 152 may be configured to store data received externally, for example, from an external device or through manual input. In one embodiment, output device 150 may include a transceiver 153 for wirelessly transmitting data to and from an external device, for example a cell phone, PDA, a wireless network, and the like. In a particular embodiment, data stored in the storage device 152 may be transmitted to an external device by the transciever 153.

Input device 190 may include a keypad 191, a switch 192, and a transciever 193. Transciever 193 may be similar to the transciever 153. Accordingly, transciever 193 may be configured to transmit data to and from an external device. Keypad 191 and switch 192 may be provided to provide manual input to the absorbent article 110, for example, to enter an age of the user, health data of the user, to start a timer 116, and the like.

As illustrated in FIG. 1B, a battery 160 may provide power to operate the output device 150, input device 190, and the processing circuit 140. Accordingly, the output device 150, input device 190, and the processing circuit 140 may be coupled with the battery 160, as illustrated. As discussed above, in some embodiments, the battery 160 electrodes may be also be configured to perform the function of the sensor electrodes 130. Accordingly, signal conditioning circuit 141 may receive input from the battery 160 to receive one or more electrical signals therefrom.

### Fluid Activated Batteries

Embodiments of the invention provide absorbent articles that do not require a constant source of power. As discussed above, a battery 160 that is activated by fluid may be provided, thereby obviating the prior art problems with battery self discharge. Furthermore, forming the fluid activated battery 160 in the absorbent article may be much cheaper and smaller than including conventional batteries, thereby making them suitable for use with disposable absorbent articles.

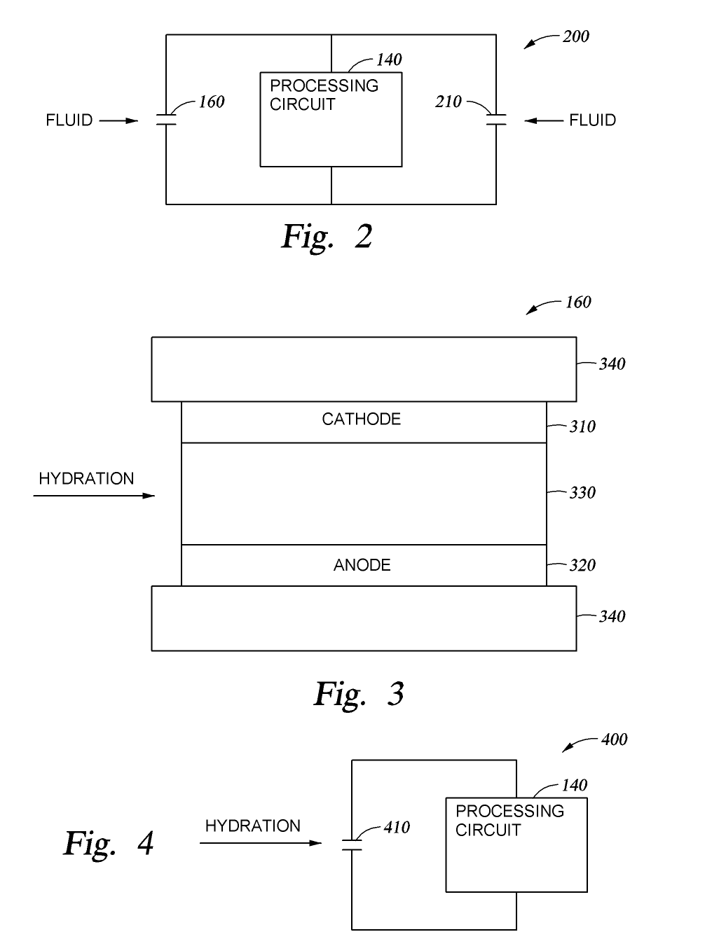

FIG. 2 illustrates a sensor circuit 200, according to an embodiment of the invention. As illustrated in FIG. 2, sensor circuit 200 may include a pair of sensor electrodes 210, battery 160, and a processing circuit 140. As discussed earlier processing circuit 140 may be configured to determine an electrical property of the sensor electrodes 210. For example, in one embodiment, processing circuit 140 may be configured to determine a resistance across the sensor electrodes 210.

The sensor electrodes 210 may be formed on an insulative substrate, such as silicon, fused silicon dioxide, silicate glass, alumina, aluminosilicate ceramic, an epoxy, an epoxy composite such as glass fiber reinforced epoxy, polyester, polyimide, polyamide, polycarbonate, etc. Sensor electrodes 210 may be formed in any shape and dimensions on the substrate of the substrate and may include a detection working electrode, a counter electrode, and a reference electrode. The electrodes 210 may be positioned at any angle to permit the flow of hydration between the electrodes. In one embodiment, the reference and counter electrodes may be combined into a single “pseudo” electrode.

The detection working electrode is typically formed from a thin film of conductive material disposed on an insulating substrate. Generally speaking, a variety of conductive materials can be used to form the detection working electrode. Suitable materials include, for example, carbon, metals, metal-based compounds (e.g., oxides, chlorides, etc.), metal alloys, conductive polymers, combinations thereof, and so forth. Examples of carbon electrodes include glassy carbon, graphite, mesoporous carbon, nanocarbon tubes, fullerenes, etc. Commercially available carbon paste may also be used.

Examples of metals that may be suitable for the current invention include platinum, palladium, gold, tungsten, titanium, etc, and their alloys. Certain metal paste compositions may also be used for the construction of the working electrodes. Thin films of these materials can be formed by a variety of methods including, for example, sputtering, reactive sputtering, physical vapor deposition, plasma deposition, chemical vapor deposition, printing, and other coating methods. For instance, carbon or metal paste based conductive materials are typically formed using screen printing, which either can be done manually or automatically. Likewise, metal-based electrodes may be formed using standard sputtering or CVD techniques, or by electrochemical plating.

Discrete conductive elements may be deposited to form each of the detection working electrode, for example, using a patterned mask. Alternatively, a continuous conductive film may be applied to the substrate and then the detection working electrode can be patterned from the film. Patterning techniques for thin films of metal and other materials may include photolithographic techniques. An exemplary technique includes depositing a thin film of conductive material and then depositing a layer of a photoresist over the thin film. Typical photoresists are chemicals, such as organic compounds, that are altered by exposure to light of a particular wavelength or range of wavelengths. Exposure to light makes the photoresist either more or less susceptible to removal by chemical agents. After the layer of photoresist is applied, it is exposed to light, or other electromagnetic radiation, through a mask.

Alternatively, the photoresist is patterned under a beam of charged particles, such as electrons. The mask may be a positive or negative mask depending on the nature of the photoresist. The mask may include the desired pattern of working electrodes. Once exposed, the portions of the photoresist and the thin film between the working electrodes is selectively removed using, for example, standard etching techniques (dry or wet), to leave the isolated working electrodes of the array.

The detection working electrode may have a variety of shapes, including, for example, square, rectangular, circular, ovoid, and so forth. The detection working electrode may have varying dimensions (e.g., length, width, or diameter). The surface smoothness and layer thickness of the electrode may be controlled through a combination of a variety of parameters, such as mesh size, mesh angle, and emulsion thickness if using a printing screen. Emulsion thickness can be varied to adjust wet print thickness. The dried thickness may be slightly less than the wet thickness because of the vaporization of solvents.

In one embodiment, the sensor electrodes may be physically separated by an absorbent material. For example, in one embodiment, the material used to form an absorbent area 120 may physically separate the sensor electrodes 210. When no fluid is present between the sensor electrodes, processing circuit 140 may detect a high resistance, or an open circuit between the sensor electrodes 210. When fluid is received in the absorbent article, the fluid may soak the absorbent material between the sensor electrodes 210 and electrically connect the electrodes. Therefore processing circuit 140 may detect a change in an electrical property, for example, resistance, between the electrodes, thereby allowing the processing circuit 140 to detect a wetting event.

Battery 160 may be configured to provide power to the processing circuit 140. Accordingly, battery 160 may be an electrochemical cell for converting chemical energy into electrical energy. As discussed earlier, battery 160 may be activated when fluid is received in the absorbent article. The battery portion of the sensor circuit may be formed on an insulative substrate, such as silicon, fused silicon dioxide, silicate glass, alumina, aluminosilicate ceramic, an epoxy, an epoxy composite such as glass fiber reinforced epoxy, polyester, polyimide, polyamide, polycarbonate, etc. Battery 160 may also be directly formed in the absorbent article, for example, a diaper. Battery electrodes, in any shape and dimensions may be formed on the absorbent area 120. Specifically, a cathode, an anode, and an electrolyte zone, may formed on the absorbent area in an area likely to receive fluid. These electrodes may be positioned at any angle to the flow of the fluid through a fluid guide. In one embodiment of the invention, the battery 160 may be a series of smaller batteries for larger capacity and higher voltage.

FIG. 3 illustrates a detailed view of the battery 160 according to one embodiment of the invention. As illustrated in FIG. 3, battery 160 may include a pair of electrodes. Specifically, battery 160 may include a cathode 310 and anode 320. In one embodiment, cathode 310 may be formed from a material that is capable of releasing electrons. Suitable materials include, for example, metals, metal-based compounds (e.g., oxides, chlorides, etc.), metal alloys, polymers, combinations thereof, and so forth. Anode 320, on the other hand, may be formed from a material that is capable of accepting electrons. Examples of anodes, for example, may include metals, metal-based compounds (e.g., oxides, chlorides, etc.), metal alloys, polymers, carbon (graphite, malodorous carbon, halocarbon tubes, fullerenes, etc), combinations thereof, and so forth.

In one embodiment of the invention, cathode 310 and anode 320 may be formed by using two different metal/metal salts redox couples with different redox potentials. In another embodiment, two electrodes may be formed by pure metals with different redox potentials when the two electrodes are connected by an electrolyte with ions that can complete the electrode redox couples. Examples of metals used may include platinum, palladium, gold, tungsten, titanium, etc, and their alloys. Thin films of such metals may be formed on an absorbent area 120 by a variety of methods including, for example, sputtering, reactive sputtering, physical vapor deposition, plasma deposition, chemical vapor deposition, printing, and other coating methods. For example, carbon or metal paste based conductive materials may be formed using screen printing, which either may be done manually or automatically. Likewise, metal-based electrodes may be formed using standard sputtering or CVD techniques, or by electrochemical plating.

As illustrated in FIG. 3, a spacer material 330 may physically separate the cathode 310 from the anode 320. Spacer material 330 may be any suitable porous material configured to soak up hydration, for example, filter paper. In one embodiment of the invention, the spacer material 330 may be formed from the same material as the absorbent area 120.

The components of battery 160, for example, the cathode 310, anode 320, and the spacer material 330 may be encapsulated in a suitable insulator material 340, for example, plastic. Insulator material 340 may be configured to prevent a person wearing the absorbent article from touching components of the battery 160, thereby preventing the person from receiving an electric shock. Insulator material 340 may be formed such that hydration received in the absorbent article 110 is transferred to the battery 160.

In one embodiment of the invention, cathode 310 and anode 320 may be made from materials with different reduction (redox) potentials. A redox potential may define the tendency of a material to acquire electrons, and therefore be reduced. For example, in one embodiment, cathode 310 may be made from magnesium and anode 320 may be made from copper/copper(I) chloride.

In one embodiment, spacer material 330 may include a suitable dry electrolyte material therein. The electrolyte material selected may depend on the particular metals used to form the cathode 310 and the anode 320. For example, if copper is used to form a battery electrode, copper chloride may be used as the electrolyte material. An electrolyte is an ionic conductor and an electronic insulator that may not react with the components of battery 160 other than to accept and/or donate the working ions from/to the cathode 310 and anode 320. In some embodiments, electrolytes may not be preloaded in the absorbent article. In such embodiments, fluid received in the absorbent article, for example, urine may function as an electrolyte for the battery 160.

When fluid is received in the spacer material 330, the fluid may dissolve the electrolyte contained therein. The dissolved electrolyte may interact with the cathode and anode to produce electricity. As a result of the flow of electrons, and different redox potentials of the cathode and anode, a voltage may be generated between the cathode 310 and anode 320 to power the processing circuit 140 and other circuits and devices.

The operating voltage (V) of the battery 160 across the cathode 310 and anode 320 may be defined by equation 1 below:

V=V_{oc}−IR (1)

wherein V_{oc} is the open circuit voltage, I represents the current flow through the electrolyte and R is the battery internal resistance. The open circuit voltage V_{oc}, may be defined by equation 2 below:

V_{oc}=(μₐ−μ_{c})/(−nF) (2)

wherein (μₐ−μ_{c}) is the difference in the electrochemical potential of the anode and cathode, n is the number of electrons involved in the chemical reaction of the cell, and F is Faraday's constant.

The performance of battery 160 may be defined by its physical and electrical properties. Exemplary physical properties may include the battery volume and weight. Exemplary electric properties may include voltage, capacity, energy, energy density, cycle life, and the like. Physical values of battery 160 may be selected to achieve a desired cell energy density. The energy density is a common measure used for evaluating battery systems. Energy stored in a battery may be measured by discharging a battery at an appropriate current. The energy in Watt-hour (Wh) is the product of cell's average operating voltage in Volt (V) and discharge capacity in Ampere-hour (Ah). For a given cell with the known volume (cm³) and weight (g), its energy density may be expressed as the watt hour per volume (W h I⁻¹) or watt hour per weight (W h kg⁻¹). For example, a 3.6 V cell with 450 mAh discharge capacity, 7.5 cm³ volume and 18.0 g weight, its energy density can be calculated as 216 W h I⁻¹ or 90 W h kg⁻¹.

In one embodiment of the invention, fluid received between a cathode 310 and anode 320 of a battery 160 may activate the battery and cause the battery 160 to power the processing circuit 140. Processing circuit 140 may then monitor an electrical property of sensor electrodes, for example, the sensor electrodes 210 to determine presence of fluid in the absorbent article and one or more characteristics of the fluid. For example, fluid received in the absorbent article may alter the resistance measured across the sensor electrodes. In response to detecting a change in resistance across the sensor electrodes, processing circuit 140 may be configured to generate a signal to the output device 150 to indicate a wetting event.

In one embodiment, the processing circuit 140 may be configured to generate a signal indicating a wetting event in response to being powered by the battery 160. The signal generated by the processing circuit may be enhanced by the sensor electrodes 210. For example, referring back to FIG. 2, fluid received between the cathode and anode of battery 160 may cause the battery 160 to power the processing circuit 140. Processing circuit 140 may generate a signal to an output device 150 in response to being powered by the battery 160.

The fluid may also be received between the sensor electrodes 210 and may alter an electrical property of the electrodes 210. For example, the fluid may electrically couple the sensor electrodes 210. The alteration of the electrical property of the electrodes 210 may cause the signal generated by the processing circuit 140 to become enhanced. For example, a larger current or voltage signal may be generated by the processing circuit 140 to the output device 150. In one embodiment, output device 150 may be configured to generate an audible signal. Accordingly, when an enhanced signal is received from the processing circuit 140, output device 150 may be configured to generate a louder aural signal. Therefore, the sensor electrodes may serve as a mechanism for confirming the presence of fluid in the absorbent article.

In one embodiment, output device 150 may be configured to indicate a wetting event only if an enhanced signal is received from the processing circuit 140. For example, processing circuit 140 may be powered by a battery 160 when a wetting event occurs. However, in one embodiment, an indication regarding the presence of fluid in the absorbent article may be sent only if the wetting event is also detected by the sensor electrodes 210. Therefore, the sensor electrodes 210 may serve as a means for confirming the presence of fluid in the absorbent article, thereby avoiding the generation of a false wetness indication by the powering of the processing circuit 140 by the battery.

For example, it is possible that the battery may generate electrical power by receiving fluid, for example, by the sweating of a user wearing the absorbent article. However, if the absorbent article is a diaper, only the detection of urination may be desired. Accordingly, a wetness indication may not be generated unless the sensor electrodes also detect the wetness event. By providing separate electrodes to confirm the presence of fluid in the absorbent article, the generation of false wetness indications may be avoided.

In one embodiment of the invention, battery 160 may serve dual functions including powering the processing circuit 140 and detecting the presence and/or amount of analytes in fluid in the absorbent article. Accordingly, the need for a separate pair of sensor electrodes may be obviated. FIG. 4 illustrates an exemplary sensor circuit 400 wherein the battery 410 provides power to the processing circuit and also detects presence and/or amount of analytes in the fluid in an absorbent article.

As illustrated in FIG. 4, sensor circuit 400 may include a battery 410 and a processing circuit 140. Battery 410 may be similar to the battery 160 illustrated in FIG. 3. In other words, battery 410 may be configured to power processing circuit 140 in response to receiving fluid between the cathode and anode of the battery 410. In the embodiment illustrated in FIG. 4, the activation of the battery itself may serve as an indication of a wetting event occurring in the absorbent article. Accordingly, fluid received in the absorbent article may cause the battery 410 to power the processing circuit 140. In response to receiving power from the battery 410, processing circuit 140 may be configured to generate a signal to output device 150, and cause output device 150 to indicate a wetting event.

In one embodiment of the invention, the battery electrodes illustrated in FIG. 3 (cathode 310 and anode 320) and/or the sensor electrodes 210 illustrated in FIG. 2 (hereinafter collectively referred to as electrodes) may be configured to detect the presence and/or amount of specific analytes in fluid received in the absorbent article. For example, in one embodiment, one or more enzymes may be placed at or near the electrodes to catalyze a redox reaction. In other words, if an analyte is present in the fluid, the enzymes may interact with the analyte and enhance the transfer of electrons between the electrodes, thereby indicating the presence of the analyte in the fluid. Detecting the presence and/or amount of particular analytes may be particularly useful for retrieving health diagnostic data. For example, in a particular embodiment, a diaper may include a battery configured to determine the glucose levels of a person wearing the diaper based on the urine received in the diaper, thereby allowing monitoring of diabetic patients.

Enzyme substrates may be either coated on electrodes or loaded close to the electrodes so that the substrates can be dissolved in the fluid and brought close to the electrodes. In the presence of an enzyme, an enzyme catalyzed redox reaction may occur which increases electron transfer rate and boosts the current generation by a battery 160. The substrate may be permanently immobilized on the electrodes or pre-dried closely to the electrodes and may later be dissolved by the fluid to participate in the redox reaction. For example, the substrate may be pre-dried in a hydrophilic porous material, such as for example, an absorbent area 120 that may be used to introduce the fluid to the electrodes. The examples of redox enzymes include glucose oxidase and dehydrogenase for glucose, which may be used to detect glucose concentration in urine.

In one embodiment of the invention, a reagent may be sandwiched in an absorbent material physically separating a pair of electrodes. For example, in one embodiment, the reagent may be placed in a spacer material 330 of a battery 160. The enzymes present in the spacer material 330 may undergo a redox reaction with an analyte present in fluid received in the spacer material 330 and enhance the transfer of electrons between electrodes. For example, in a particular embodiment, the reagent used may be an aromatic amine. The aromatic amine may react with an analyte, for example, nitrites in urine to indicate urinary tract infection. The aromatic amine may react with an analyte, for example, nitrites in urine to indicate urinary tract infection.

In one embodiment of the invention, processing circuit 140 may be configured to detect a wetting event and the presence and/or amount of an analyte in the fluid received in an absorbent article. For example, when fluid is received, the fluid may activate a battery 160 and cause the processing circuit to be powered. Processing circuit 140 may generate a signal to indicate the presence of fluid.

Furthermore, if a particular analyte is present in the fluid, processing circuit 140 may detect a corresponding enhanced transfer of electrons, and provide an appropriate output signal to the output device 150 to indicate the presence and/or amount of the analyte. For example, in one embodiment, processing circuit 140 may display the name of a detected analyte in an LCD screen. Alternatively, processing circuit 140 may cause a recommended course of action, for example, “feed patient”, based on the rate of transfer of electrons between the electrodes.

In one embodiment, processing circuit 140 may be configured to determine the amount of the analyte present in the fluid. For example, the amount of analyte present in the fluid may be determined based on the strength of current generated between the electrodes. Accordingly, processing circuit 140 may be configured to determine and display the amount of analyte in fluid received in the absorbent article based on current measurement across the electrodes.

In one embodiment of the invention, the battery electrodes illustrated in FIG. 3 (cathode 310 and anode 320) and/or the sensor electrodes 210 illustrated in FIG. 2 (hereinafter collectively referred to as electrodes) may be configured to detect the presence and/or amount of specific ions in fluid received in the absorbent article. Accordingly, the electrodes may be coated with ion selective layers, such as thin films and coatings. Examples of ion selective materials include ion selective porous glass and crown ether derivatized materials. The materials can be physically coated or covalently attached to the electrodes.

The current generated between the electrodes may be proportional to the concentration of specific ions in fluid received between the electrodes. Therefore, the current flowing between the electrodes may be used to measure or approximate the number of ions, ion strength, pH value of the fluid, and the like. Detection of specific ions in fluid, for example, urine, may allow determination of the hydration status of a person wearing an absorbent article.

As discussed above, an electrolyte present between battery electrodes may serve serves as a media movement for charge between the battery electrodes. The effectiveness of the electrolyte may be defined by its conductivity. In one embodiment of the invention, the conductivity in electrolyte may be caused by the movement of ions such as sodium ions in a urine sample.

Ionic conductivity (σ), like electronic conductivity, may be expressed as a product of a carrier charge (q), concentration (numbers of ions per unit volume, n), and mobility of ions (the average velocity of a carrier due to an applied electric field of unit strength (b) as shown by equation 4 below:

σ=qnb (4)

The total ionic conductivity may be obtained as the sum of the contributions of all free ions in the hydration received between electrodes, as shown in equation 5 below:

σ=Σqᵢnᵢb_{I}=Σ|zᵢ|cᵢλ_{I} (5)

where the z_{I} is the charge number of the ion and λ_{I} is the equivalent conductivity, which is related to the specific conductivity as shown in equation 6 below:

λ_{I}=σ/|zᵢ|cₗ=Fb_{I} (9)

where F is Faraday's constant. Therefore, the presence of certain ions in the hydration may enhance the flow of current between electrodes, which may be detected by a processing circuit 140. If enhanced conductivity is detected processing circuit 140 may be configured to indicate presence of ions in the fluid in an output device 150. Furthermore, processing circuit 140 may be configured to determine and display the amount of the particular ion based on the strength of current between the electrodes.

### CONCLUSION

By providing a fluid activated battery, embodiments of the invention allow cheap and reliable batteries to be incorporated into disposable absorbent products. The battery may be activated only upon the receipt of fluid in the absorbent product. Therefore, the fluid activated battery may be used to detect the presence of fluid in the absorbent product. The fluid activated battery may be further configured to detect specific substances, for example specific ions or specific analytes that may be useful in diagnosing a health status of a person wearing the absorbent product.

While the foregoing is directed to embodiments of the present invention, other and further embodiments of the invention may be devised without departing from the basic scope thereof, and the scope thereof is determined by the claims that follow.

What is claimed is:

1. An apparatus, comprising:
an absorbent article; and
a sensor circuit disposed on the absorbent article and configured for detecting fluid in the absorbent article, the sensor circuit comprising:
a pair of sensor electrodes, wherein each electrode is coupled with an absorbent material, the absorbent material being configured to receive fluid to electrically couple the sensor electrodes;
a processing circuit coupled with the sensor electrodes and configured to detect the presence of fluid that electrically couples the sensor electrodes; and
a battery for providing power to the processing circuit, wherein the battery is configured to be activated by the fluid received in the absorbent article.

2. The apparatus of claim 1, wherein the battery comprises a cathode and an anode, wherein the cathode and the anode are made from materials having different redox potentials.

3. The apparatus of claim 2, wherein the cathode and anode are separated by a spacer material wherein the spacer material is an absorbent material configured to receive fluid.

4. The apparatus of claim 3, wherein the spacer material comprises an electrolyte material therein.

5. The apparatus of claim 4, wherein fluid received in the spacer material is configured to dissolve the electrolyte and facilitate electron transfer, thereby causing a voltage to be generated between the cathode and anode.

6. The apparatus of claim 1, wherein the processing circuit is further configured to generate a signal to an output device, wherein the output device is configured to indicate the presence of fluid in the absorbent article in response to receiving the signal.

7. The apparatus of claim 6, wherein the processing circuit is configured to generate the signal in response to being powered by the battery.

8. The apparatus of claim 7, wherein the fluid that electrically couples the sensor electrodes is configured to enhance the signal.

9. The apparatus of claim 1, wherein the absorbent material comprises a reagent configured to react with a predetermined analyte in the fluid and enhance transfer of electrons between the electrodes to indicate presence of the analyte.

10. The apparatus of claim 9, wherein the reagent comprises a redox enzyme configured to catalyze the redox reaction of an analyte substrate.

11. The apparatus of claim 9, wherein the processing circuit is configured to determine the amount of analyte in the fluid.

12. The apparatus of claim 1, wherein the sensor electrodes are ion selective electrodes configured to detect the presence of predetermined ions in the fluid.

13. The apparatus of claim 12, wherein the processing circuit is configured to determine the amount of ions in the fluid.

14. The apparatus of claim 1, wherein the sensor electrodes are disposed on an absorbent area of the absorbent article wherein fluid is most likely to be received.

15. The apparatus of claim 1, wherein the battery is disposed on an absorbent area of the absorbent article wherein fluid is most likely to be received.

16. An apparatus, comprising:
an absorbent article; and
a sensor circuit disposed on the absorbent article, the sensor circuit comprising:
a battery comprising at least two electrodes coated with a material for detecting the presence of analytes in fluid received in the absorbent article, wherein the battery is activated by fluid received in the absorbent article; and
a processing circuit, wherein the processing circuit is configured to receive electric power from the battery when the battery is activated, and generate a signal indicating presence of one or more predetermined analytes in the fluid received in the absorbent article.

17. The apparatus of claim 16, wherein the at least two battery electrodes comprise a cathode and an anode, wherein the cathode and the anode are made from materials having different redox potentials.

18. The apparatus of claim 17, wherein the cathode and anode are ion selective electrodes configured to detect the presence of predetermined ions in the fluid.

19. The apparatus of claim 18, wherein the processing circuit is configured to determine the amount of ions in the fluid.

20. The apparatus of claim 17, wherein the cathode and anode are separated by a spacer material wherein the spacer material is an absorbent material configured to receive fluid.

21. The apparatus of claim 20, wherein the spacer material comprises an electrolyte material therein.

22. The apparatus of claim 21, wherein fluid received in the spacer material is configured to dissolve the electrolyte and facilitate electron transfer, thereby causing a voltage to be generated between the cathode and anode.

23. The apparatus of claim 22, wherein the spacer material further comprises a reagent configured to react with a predetermined analyte in the fluid and enhance transfer of electrons between the cathode and anode to indicate presence of the analyte.

24. The apparatus of claim 23, wherein the processing circuit is configured to determine the amount of analyte in the fluid.

25. The apparatus of claim 16, wherein the processing circuit is configured to generate a signal to an output device in response to being powered by the battery, wherein the output device is configured to indicate the presence of fluid in the absorbent article in response to receiving the signal from the processing circuit.

26. The apparatus of claim 16, wherein the battery is disposed on an absorbent area of the absorbent article wherein fluid is most likely to be received.

27. A method for detecting the presence of fluid in an absorbent article, comprising:
disposing a pair of battery electrodes in an absorbent area of the absorbent article, wherein the battery electrodes are configured to generate electrical power in response to receiving fluid in the absorbent article; and
disposing a processing circuit configured to receive the electrical power from the battery electrodes and generate a signal indicating presence of one or more analytes in the fluid received in the absorbent article.

28. The method of claim 27, further comprising disposing a pair of sensor electrodes in the absorbent area, the pair of sensor electrodes being coupled with the processing circuit, wherein the fluid received in the absorbent area electrically couples the pair of sensor electrodes and enhances the signal generated by the processing circuit.

29. The method of claim 28, wherein the sensor electrodes comprise a reagent configured to react with a predetermined analyte in the fluid and enhance transfer of electrons between the electrodes to indicate presence and/or amount of the analyte.

30. The method of claim 27, wherein the battery electrodes comprise a cathode and an anode, wherein the cathode and anode are made from materials having different redox potentials.

31. The method of claim 30, wherein the cathode and anode are separated by a spacer material comprising an electrolyte material therein, wherein the spacer material is an absorbent material configured to receive fluid.

32. The method of claim 31, wherein the spacer material further comprises a reagent configured to react with a predetermined analyte in the fluid and enhance transfer of electrons between the cathode and anode to indicate presence of the analyte.

33. The method of claim 32, wherein the processing circuit is configured to determine the amount of analyte present in the fluid.

34. The method of claim 27, wherein the battery electrodes are ion selective electrodes configured to detect the presence of predetermined ions in the fluid.

35. The method of claim 34, wherein the processing circuit is configured to determine the amount of ions present in the fluid.

36. An apparatus, comprising:
an absorbent article; and
a sensor circuit disposed on the absorbent article, the sensor circuit comprising:
a battery comprising at least two electrodes separated by an absorbent material comprising a material for detecting the presence of analytes in fluid received in the absorbent article, wherein the battery is activated by fluid received in the absorbent article; and
a processing circuit, wherein the processing circuit is configured to receive electric power from the battery when the battery is activated and generate a signal indicating presence of one or more predetermined analytes in the fluid received in the absorbent article.
